(19) United States
(12) Patent Application Publication
(10) Pub. No.: US 2024/0366302 A1
(43) Pub. Date: Nov. 7, 2024

(54) SYSTEM AND METHOD FOR LASER HAIR TREATMENT

(71) Applicant: LUMENIS BE LTD., Yokneam (IL)

(72) Inventors: Amir Kishon, Yokneam (IL); Sachi Norman, Yokneam (IL); Zoya Cahana, Yokneam (IL); Karin Srage, Yokneam (IL); Assaf Preiss, Shimshit (IL); Moshe Elazar, Kadima-Tzoran (IL); Kalia Mendel, Yokneam (IL)

(21) Appl. No.: 18/648,431

(22) Filed: Apr. 28, 2024

Related U.S. Application Data
(60) Provisional application No. 63/463,969, filed on May 4, 2023.

(51) Int. Cl. A61B 18/20; A61B 18/00

(52) U.S. Cl. CPC A61B 18/203; A61B 2018/00476; A61B 2018/2065

(57) ABSTRACT

A method and system for hair treatment comprising a laser module configured for generating non-ablative laser; a handpiece connected to the laser module configured to receive the non-ablative laser and deliver a fractional pattern of the non-ablative laser to a treatment site on a person's scalp. A controller for controlling parameters of the non-ablative laser to apply the hair and activating the laser module for a predetermined treatment time, at a predetermined pulse rate, and a predetermined pulse duration. Delivering, by the handpiece, the non-ablative laser to the treatment site on the person's scalp; and deactivating, by the controller, the laser module.

### TECHNOLOGICAL FIELD

The present invention is in the aesthetic field and relates specifically to hair treatment, and more specifically to improving scalp hair condition.

### BACKGROUND

Hair loss, or alopecia, is a major problem that affects the majority of the population, both men and women, at some point in their life, and can be caused by a variety of conditions. Alopecia is defined as a common condition in which the hair fails to adequately cover the scalp, and thus, can cause psychosocial distress.

Common baldness, otherwise known as Androgenic Alopecia, is the most common type of non-scarring alopecia, and is characterized by the miniaturization of hair follicles (HF), shortened anagen and prolonged telogen hair growth cycle phases.

Numerous treatment solutions are on the market, including non-invasive and invasive treatments. The invasive treatments include surgical procedures which can be accompanied with pain and inflammation risks. The non-invasive or minimally-invasive treatments include, for example, injectables, topical treatments, supplement oral treatments and treatments by energy, such as optical energy. At least some of these treatments are ineffective, either totally or for long-lasting benefits. Topical corticosteroids are often the first line of treatment for mild patchy alopecia. However, this method cannot be used for rapidly progressing variants and does not prevent hair loss at other sites. Furthermore, both topical and intralesional steroids increase the risk of cutaneous atrophy at the site of treatment, and intralesional steroids may decrease bone mineral density. Systemic corticosteroids have also had promising results, with one study demonstrating 62% of patients exhibiting full hair regrowth. However, the therapy can cause substantial adverse side effects such as acne, myalgias, osteoporosis, numbness, and weight gain.

### SUMMARY OF INVENTION

In at least one aspect of the invention, there is a method for hair treatment comprising: providing a laser module configured for generating a non-ablative laser beam; providing a handpiece connected to the laser module configured to receive the non-ablative laser beam, and deliver a fractional pattern of the non-ablative laser beams to a treatment site on a person's scalp; providing a controller adapted for controlling parameters of the non-ablative laser beam to apply the hair; activating, by the controller, the laser module for a predetermined treatment time, at a predetermined pulse rate, and a predetermined pulse duration; delivering, by the handpiece, the non-ablative laser to the treatment site on the person's scalp; and deactivating, by the controller, the laser module.

In another aspect of the invention, the method wherein the laser module generates the non-ablative laser beam with a wavelength in the range of 1000-2000 nm and wherein the laser module generates the non-ablative laser beams having at least one of the following wavelengths: 1940 nm, 1927 nm, 1565 nm, 1550 nm, 1540 nm, 1470 nm, 1450 nm, 1440 nm, 1410 nm, 1340 nm, 1320 nm, 1064 nm and 1060 nm. The method further comprises providing a scanner configured define at least one of size and shape of a fractional pattern of non-ablative laser beams and delivering to the treatment site the fractional pattern of non-ablative laser beams comprising a plurality of spaced-apart micro-beams forming a certain shape that defines the treatment site. The method wherein the plurality of spaced-apart micro-beams forms at least one of the following shapes: hexagon, doughnut, circle, square, horizontal rectangle, vertical rectangle, horizontal line, and vertical line and wherein the plurality of spaced-apart micro-beams forms a circular shape having a diameter of up to 18 mm.

In one aspect of the invention, the method wherein the handpiece delivers the fractional pattern of non-ablative laser is at least one of: a density a range of 50 to 500 micro-beams per cm²; or an energy between 10-35 mJ per each micro-beam. The method further comprising, providing one more of tips configured to be mounted at a distal end of the handpiece, and the tips being configured to do at least one of the following: define the shape of the treatment site; form a spacer between a laser output of the handpiece and the treatment site; move grown hair aside for treatment; or conform to topography of the treatment site. The method further comprising, providing one more of cooling devices configured to maintain an optimal temperature of the handpiece or/and a tip mounted on a distal end of the handpiece.

In yet another aspect of the method, it further comprises: providing temperature sensors; and automatically activating, by the controller, the one or more cooling devices if at least one of the following occurs: the energy level is above a predetermined energy; the density level is above a predetermined density; and a temperature of the handpiece, a tip and/or the treatment site is above a predetermined temperature, based on the temperature sensors. The method further comprising providing a registration module configured to register location(s) of treatment sites during a treatment session.

In another aspect, there is a system for hair treatment comprising: a laser module configured for generating a non-ablative laser beam; a handpiece connected to the laser module configured to receive the non-ablative laser beam, and deliver a fractional pattern of the non-ablative laser beams to a treatment site on a person's scalp; a controller adapted for controlling parameters of the non-ablative laser beam to apply the hair; a non-transitory computer readable medium programmed with computer readable code that upon execution by the controller causes the controller to: activate the laser module for a predetermined treatment time, at a predetermined pulse rate, and a predetermined pulse duration; deliver the non-ablative laser to the treatment site on the person's scalp; and deactivate the laser module.

In a further aspect of the system, wherein the laser module generates the non-ablative laser beam with a wavelength in the range of 1000-2000 nm and wherein the laser module generates the non-ablative laser beams having at least one of the following wavelengths: 1940 nm, 1927 nm, 1565 nm, 1550 nm, 1540 nm, 1470 nm, 1450 nm, 1440 nm, 1410 nm, 1340 nm, 1320 nm, 1064 nm and 1060 nm. The system further comprises a scanner configured define at least one of size and shape of a fractional pattern of non-ablative laser beams and delivers to the treatment site the fractional pattern of non-ablative laser beams comprising a plurality of spaced-apart micro-beams forming a certain shape that defines the treatment site.

In at least one aspect the system, wherein the plurality of spaced-apart micro-beams forms at least one of the following shapes: hexagon, doughnut, circle, square, horizontal rectangle, vertical rectangle, horizontal line, and vertical line and wherein the plurality of spaced-apart micro-beams forms a circular shape having a diameter of up to 18 mm. Also, the system wherein the handpiece delivers the fractional pattern of non-ablative laser is at least one of: a density a range of 50 to 500 micro-beams per cm²; or an energy between 10-35 mJ per each micro-beam.

In a final aspect, the system further comprises one more of tips configured to be mounted at a distal end of the handpiece, and the tips being configured to do at least one of the following: define the shape of the treatment site; form a spacer between a laser output of the handpiece and the treatment site; move grown hair aside for treatment; or conform to topography of the treatment site. Also, the system further comprises one more of cooling devices configured to maintain an optimal temperature of the handpiece or/and a tip mounted on a distal end of the handpiece; temperature sensors; the non-transitory computer readable medium programmed with computer readable code that upon execution by the controller causes the controller to: automatically activate the one or more cooling devices if at least one of the following occurs: the energy level is above a predetermined energy; the density level is above a predetermined density; and a temperature of the handpiece, a tip and/or the treatment site is above a predetermined temperature, based on the temperature sensors.

### BRIEF DESCRIPTION OF THE DRAWINGS

FIG. 1 illustrates a diagram of a system of at least some embodiments of the present disclosure.

FIG. 2 is a flow diagram of the method of at least some embodiments of the present disclosure.

FIG. 3 illustrates the regeneration of hair follicles under treatment(s) carried out with the presently disclosed subject matter of at least some embodiments of the present disclosure.

### GENERAL DESCRIPTION

The presently disclosed subject matter relates to non-invasive systems and methods for hair treatment, specifically improvement of scalp hair appearance, inter alia, through new hair growth on the scalp and/or reducing or preventing hair loss. A fractional laser treatment that is applied to the scalp causes localized tissue coagulation that triggers a wound healing response that recruits the body's mechanisms of cell growth to regenerate the skin and adnexal structures such as hair follicles in a favorable fashion. In some embodiments, treatments are provided with lasers and monochromatic light, for example, with the use of a 308-nm excimer laser, a 904-nm pulsed Infrared diode laser, or with ae neodymium: yttrium aluminum garnet laser (ND: YAG) to treat alopecia are employed.

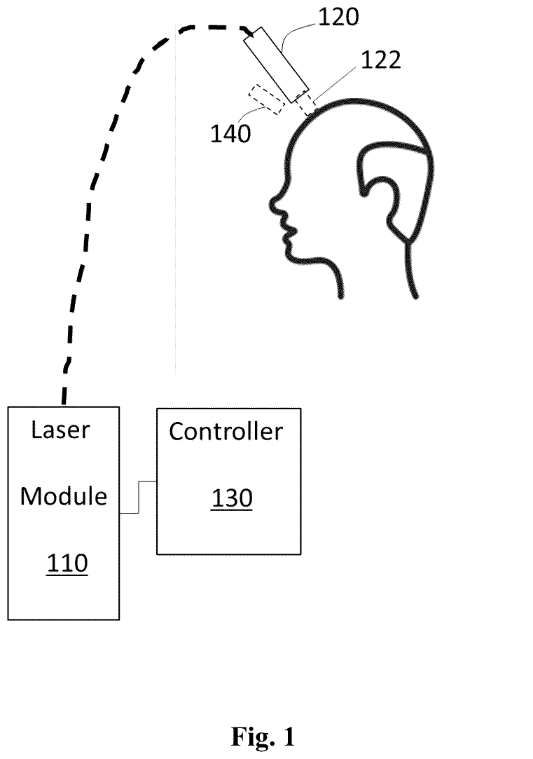

Reference is now made to FIG. 1 illustrating, by way of a block diagram, a non-limiting exemplary embodiment of a system 100 according to the presently disclosed subject matter. Dashed lines indicate optional elements. The system 100 is operable for applying treatment to improve appearance of scalp hear, e.g. by promoting hair growth and/or reducing hair loss. The system includes a laser module 110 configured and operable to generate a non-ablative laser for use in the hair treatment. A handpiece 120 connected in optical communication with the laser module, either being directly connected to the laser module or via an optical fiber and configured to receive the non-ablative laser. The handpiece may further be adapted to deliver a fractional pattern of the non-ablative laser to a treatment site on a person's scalp. In some embodiments, a controller 130 is provided in communication with the laser module and the handpiece and may be configured to control one or more parameters of the laser module or/and the handpiece to apply the hair treatment, thereby improving appearance of the hair. In some embodiments, the system includes non-transitory computer readable medium (not shown) programmed with computer readable code.

In some embodiments, the laser module of the system is configured for generating the non-ablative laser having a wavelength in the range of 1000-2000 nm. In some embodiments, the laser module is configured for generating the non-ablative laser having at least one of the following wavelengths (in nm): 1940, 1927, 1565, 1550, 1540, 1470, 1450, 1440, 1410, 1340, 1320, 1064 and 1060. In some embodiments, the laser module utilizes Erbium Glass Fiber laser technology. In some embodiments, the laser module utilizes ND: YAG laser technology.

In some embodiments, the handpiece (or a probe, that can be operated by hand or by electrical or mechanical means) of the system is adapted to deliver the fractional pattern of non-ablative laser comprising a plurality of spaced-apart micro-beams forming a certain shape defining the treatment site. In some embodiments, the handpiece of the system is adapted to deliver the fractional pattern of non-ablative laser comprising a plurality of spaced-apart micro-beams forming at least one of the following shapes defining the treatment site: Hexagon, doughnut, circle, square, horizontal rectangle, vertical rectangle, horizontal line, and vertical line. In some embodiments, the handpiece of the system is adapted to deliver the fractional pattern of non-ablative laser comprising a plurality of spaced-apart micro-beams forming a circular shape having a diameter of up to 18 mm defining the treatment site.

In some embodiments, the handpiece of the system is configured to deliver the fractional pattern of a non-ablative laser comprising a plurality of spaced-apart micro-beams having a density in a range of 50 to 500 micro-beams per cm². In some embodiments, the handpiece is adapted to deliver the fractional pattern of non-ablative laser comprising a plurality of spaced-apart micro-beams each micro-beam having a treatment spot size of tens of micrometers, e.g. 50 μm, 100 μm, 150 μm, or a combination thereof. In some embodiments, the laser module and the handpiece are configured to deliver the fractional pattern of non-ablative laser having an energy between 10-35 mJ per each micro-beam. In some embodiments the energy is between 15-20 mJ per each micro-beam.

In some embodiments, the laser module system is part of a skin diagnostic and treatment system using machine learning models. In some embodiments, the skin diagnostic and treatment system analyzes captured multi-spectral images to automatically determine the treatment parameters of the laser module system, including a customized fractional pattern of spaced-apart micro-beams following, for example, the shape of the area of scalp lacking hair. In some embodiments, the skin diagnostic and treatment system analyzes a set of multi-spectral images taken right after the laser module system treatment to determine new treatment parameter and a customized fractional pattern for the laser module system.

In some embodiments, the system is adapted to deliver the non-ablative laser in pulses. In some embodiments, the pulses have a pulse duration of up to 10 ms. In some embodiments, the pulses have a pulse rate of between 0.5 to 2 Hz. In some embodiments, the system comprises a registration module (possibly within the controller) configured to register location of the treatment sites visited during a treatment session, to enable guidance to same treatment sites during a subsequent treatment session.

In some embodiments, as illustrated by dashed lines, the system includes one or more tips 122, mountable on the distal end of the handpiece. In some embodiments the one or more tips define respective treatment site area or/and shape. In some embodiments, the one or more tips form a spacer between a laser output of the handpiece and the treatment site. In some embodiments, the one or more tips are configured to clear a path of the non-ablative laser towards the treatment site on the scalp by moving aside grown hair. In some embodiments, the one or more tips are configured to conform to topography of the treatment site.

In some embodiments, as illustrated by dashed lines, the system includes one or more cooling devices 140. The cooling device(s) may be provided within the handpiece or be provided in a separate enclosure. In some embodiments, the handpiece comprises a first cooling device configured to maintain temperature of the handpiece or/and a tip mounted on a distal end thereof at an optimal temperature. In some embodiments, the optimal temperature of the tip is in the range of 7-13 degrees C°. The treatment site can be passively cooled by the tip contacting the treatment site. In some embodiments, the system comprises a second cooling device configured to cool the treatment site and keep it at an optimal temperature. Cooling the treatment site can aid in reducing or eliminating pain that might be caused by the treatment. Either the first or the second cooling devices, or both, may be provided in the system.

In some embodiments, the controller is configured to automatically activate the first and/or second cooling device when the controller detects certain energy level and/or density level above predetermined or undetermined levels. In some embodiments, the controller is configured to gradually increase the cooling intensity upon corresponding gradual increase in the energy level and/or the density level. In some embodiments, the controller is configured to automatically activate the first and/or second cooling device based on feedback from one or more temperature sensors reporting temperature of the handpiece and/or the tip and/or the treatment site.

In some embodiments, the handpiece comprises a scanner for defining at least one of size and shape of the fractional pattern output by the non-ablative laser. In some embodiments, the scanner is a galvanometric scanner.

In some embodiments, the method used in the system comprises irradiating a target portion on the scalp with a fractional non-ablative laser having a wavelength of 1565, the fractional non-ablative laser formed by a plurality of spaced-apart micro-beams having a density in a range of between 50 and 500 micro-beams per cm² and an energy of between 10-35 mJ per each micro-beam.

In order to better understand the subject matter that is disclosed herein and to exemplify how it may be carried out in practice, embodiments will now be described, by way of non-limiting examples only, with reference to the accompanying drawings, in which;

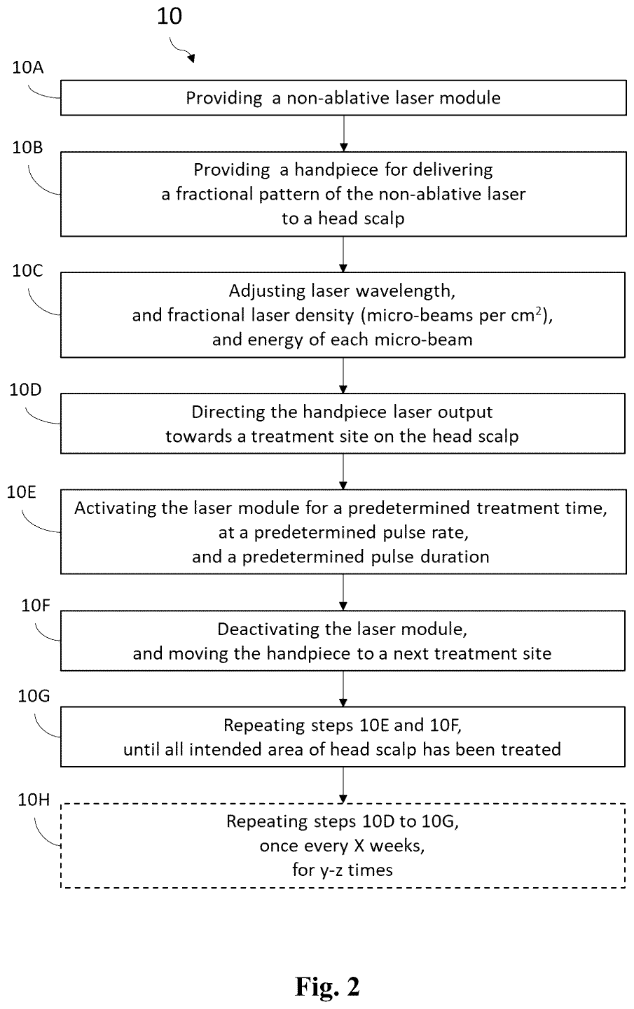

FIG. 2 illustrates, in a way of flow diagram, a non-limiting exemplary embodiment of a method 10 according to the presently disclosed subject matter. Dashed lined indicate optional steps.

At step 10A, the method comprises providing a non-ablative laser module.

At step 10B, the method comprises providing a handpiece for delivering a fractional pattern of the non-ablative laser to a head scalp. In some embodiments, the step includes providing a tip mounted on the distal end of the handpiece, as described above.

At step 10C, the method comprises adjusting laser wavelength of the laser module (e.g. 1565 nm), and fractional laser density (e.g. 350 micro-beams per cm²), and energy of each micro-beam (e.g. 17-19 mJ per micro-beam).

At step 10D, the method comprises directing the handpiece laser output towards a treatment site on the head scalp.

At step 10E, the method comprises activating the laser module for a predetermined treatment time, at a predetermined pulse rate (e.g. 0.5-2 Hz), and a predetermined pulse duration (e.g. 10 ms for each pulse).

At step 10F, the method comprises deactivating the laser module, and moving the handpiece to a next treatment site, which can be adjacent to or located at a predetermined location with respect to the previous treatment site. In some embodiments, the treatment route is planned to minimize pain resulting from the treatment. For example, the treatment stamping route can be random or semi-random. In some embodiments, the fractional pattern of the laser can be a line or narrow rectangle, for example at an area with gown hair, or rectangular/circular/hexagonal, at areas lacking grown hair.

At step 10G, the method comprises repeating steps 10E and 10F, until all intended area of head scalp has been treated.

At step 10H, the method comprises repeating steps 10D to 10G, once every X (e.g. 3) weeks, for y-z (3-6) times. In some embodiments, the method comprises a registration process recording the e previous treatment sites visited in a treatment session during steps 10E and 10F, to serve as guidance to treat the same (or a different) area of the head scalp, during the next treatment sessions according to step 10H. In some embodiments, the treatment method is repeated 3-6 times with 3-week interval between the consecutive treatments. In some embodiments, the method comprises performing each treatment session for about 20 minutes. In some embodiments, the method comprises applying the treatment in a stamping mode by contacting the treatment site with the handpiece, activating the laser module for a predetermined period, stopping the laser module, moving the handpiece to a next treatment site and starting again.

Optionally, during the treatment, the method comprises cooling the treatment site to keep it at optimal temperature while enabling increasing at least one of the energy and density of the fractional pattern, which otherwise are unbearable, and while preventing adverse effects, burns or scars, that might be caused by the intensive treatment. Cooling the treatment site can be done passively by contacting the treatment site with a tip being actively cooled (e.g., by the first cooling device described above), and/or actively by applying direct cooling, e.g. by cooled air, to the treatment site.

The applicant has performed animal studies as well as clinical studies to evaluate hair growth (e.g., as demonstrated through improvement in scalp hair appearance) in males and females utilizing treatment based on the presently disclosed subject matter.

In one clinical study, the study included one hundred and thirty-two (132) cases, with ages ranging from 21 to 80 years and a mean age of 38.2±11.5 years. Of them, in 57 (57/132, 43.2%) of the cases the documented diagnosis was Androgenic Alopecia, and in 75 cases (75/132, 56.8%) there was no documented diagnosis in the clinic records. The number of males and females' subjects was identical (66), and the majority of subjects had Fitzpatrick skin type III (79/132, 59.8%)), with some having skin types II (26/132, 19.7%) or IV (3/132, 2.3%). Skin type was not specified for 24 subjects (24/132, 18.2%).

A total of Ninety-eight (98) cases were evaluated in the study, with ages ranging from 21 to 66 years old and a mean age of 37.2±9.9 years. Of them, in 44 (44/98, 44.9%) of the cases the documented diagnosis was Androgenic Alopecia, and in 54 cases (54/98, 55.1%) there was no documented diagnosis. Females slightly outnumbered males (51 vs. 47), and the majority of subjects had Fitzpatrick skin type III (60/98, 61.2%), with some having skin types II (18/98, 18.4%) or IV (1/98, 1%). Skin type was not specified for 19 subjects. Fourteen subjects had a medical condition, all of which were endocrine disorders. Four subjects were taking concomitant medications, and only one subject had undergone previous hair growth treatment.

During the study, all hair growth treatments that were included in this study utilized a fractional non-ablative laser of 1565 nm wavelength, the density of spots per square centimeter was 350, and the energy levels ranged from 17 to 19 mJ.

The majority of patients underwent either 3 (74/132, 56.1%) or 4 (39/132, 29.5%) treatments, while a smaller number received 5 (9/132, 6.8%) or 6 (10/132, 7.6%) treatments.

No adverse reactions were reported among the 132 enrolled subjects during or following the laser treatment, indicating the safety of the treatment. The primary performance endpoint was evaluated by measuring the rate of accurate identification of the post treatment images by 3 blinded reviewers. Success was defined as correct identification of the post-treatment image by two or more blinded reviewers. For the 98 included subjects, the overall success rate was 96.9% (95% CI: 91.38%-98.95%). Among the sub-groups, the success rates were 97.7% (95% CI: 87.8%-99.7%) for subjects with Androgenic Alopecia (n=44), 96.3% (95% CI: 84.7%-99.2%) for those with no documented diagnosis (n=54), 97.9% (95% CI: 91.1%-99.7%) for male subjects (n=47), and 96.1% (95% CI: 82.5%-99.2%) for female subjects (n=51).

Based on the data presented in the retrospective, observational, single-center study, it can be concluded that the use of fractional non-ablative 1565 nm laser is safe and effective in promoting visible hair growth and improving scalp hair appearance. The effectiveness of the treatment was statistically significant, as demonstrated by the high percentage of cases classified correctly by blinded reviewers. Therefore, the findings suggest that fractional non-ablative 1565 nm laser treatment can be considered as a viable option for individuals seeking to improve hair growth.

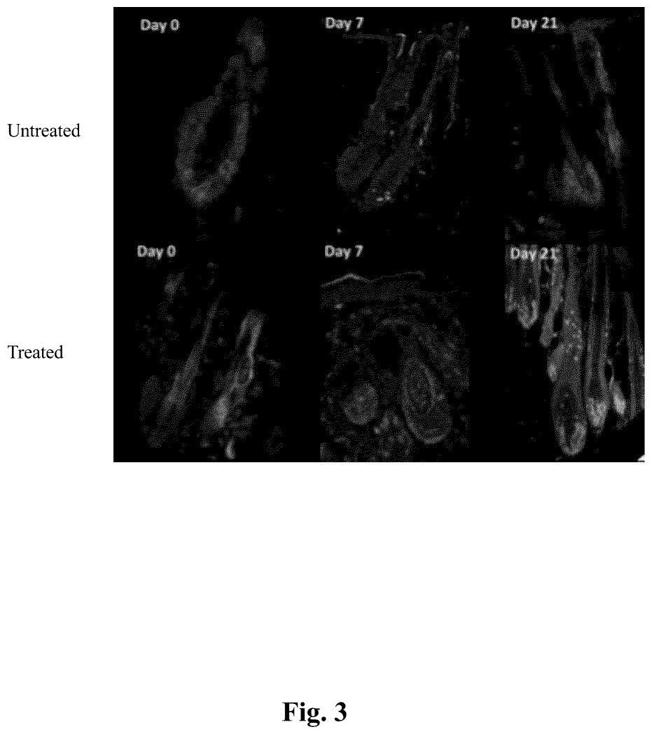

In an in-vivo animal study on mice, the applicant has found that a fractional non-ablative 1565 nm laser treatment is effective for the regeneration of hair follicles. The study included Gene expression analysis of Cytokine's expression indicative of the inflammation, which was significantly enhanced during the treatment, and Gene expression analysis of known players in hair follicle regeneration and growth, Wnt/β-cat pathway and Sonic hedgehog (HSS), which showed a significant correlation between the laser treatments, in a dose dependent manner, with energy and density effect. The study included histological analysis which showed the fractional non-ablative laser effect of tissue coagulation at day 0, infiltration of immune factors at day 7 and complete tissue repair at day 21 with the observation of complete formation of hair follicles. Immunofluorescence images illustrating the regeneration of the hair follicles under treatment carried out with the presently disclosed subject matter are shown in FIG. 3.

Various detailed embodiments of the present disclosure, taken in conjunction with the accompanying figures, are disclosed herein; however, it is to be understood that the disclosed embodiments are merely illustrative. The aforementioned examples are, of course, illustrative and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure. In addition, throughout the specification, the meaning of “a,” “an,” and “the” include plural references. The meaning of “in” includes “in” and “on.”

1. A method for hair treatment comprising:
providing a laser module configured for generating a non-ablative laser beam;
providing a handpiece connected to the laser module configured to receive the non-ablative laser beam, and deliver a fractional pattern of the non-ablative laser beams to a treatment site on a person's scalp;
providing a controller adapted for controlling parameters of the non-ablative laser beam to apply the hair;
activating, by the controller, the laser module for a predetermined treatment time, at a predetermined pulse rate, and a predetermined pulse duration;
delivering, by the handpiece, the non-ablative laser to the treatment site on the person's scalp; and
deactivating, by the controller, the laser module.

2. The method of claim 1, wherein the laser module generates the non-ablative laser beam with a wavelength in the range of 1000-2000 nm.

3. The method of claim 2, wherein the laser module generates the non-ablative laser beams having at least one of the following wavelengths: 1940 nm, 1927 nm, 1565 nm, 1550 nm, 1540 nm, 1470 nm, 1450 nm, 1440 nm, 1410 nm, 1340 nm, 1320 nm, 1064 nm and 1060 nm.

4. The method of claim 1, further comprises providing a scanner configured define at least one of size and shape of a fractional pattern of non-ablative laser beams and delivering to the treatment site the fractional pattern of non-ablative laser beams comprising a plurality of spaced-apart micro-beams forming a certain shape that defines the treatment site.

5. The method of claim 4, wherein the plurality of spaced-apart micro-beams forms at least one of the following shapes: hexagon, doughnut, circle, square, horizontal rectangle, vertical rectangle, horizontal line, and vertical line.

6. The method of claim 5, wherein the plurality of spaced-apart micro-beams forms a circular shape having a diameter of up to 18 mm.

7. The method of claim 4, wherein the handpiece delivers the fractional pattern of non-ablative laser is at least one of: a density a range of 50 to 500 micro-beams per cm²; or an energy between 10-35 mJ per each micro-beam.

8. The method of claim 1, further comprising, providing one more of tips configured to be mounted at a distal end of the handpiece, and the tips being configured to do at least one of the following:
define the shape of the treatment site;
form a spacer between a laser output of the handpiece and the treatment site;
move grown hair aside for treatment; or
conform to topography of the treatment site.

9. The method of claim 1, further comprising, providing one more of cooling devices configured to maintain an optimal temperature of the handpiece or/and a tip mounted on a distal end of the handpiece.

10. The method of claim 9, further comprising,
providing temperature sensors; and
automatically activating, by the controller, the one or more cooling devices if at least one of the following occurs:
the energy level is above a predetermined energy;
the density level is above a predetermined density; and
a temperature of the handpiece, a tip and/or the treatment site is above a predetermined temperature, based on the temperature sensors.

11. The method of claim 1, further comprising providing a registration module configured to register location(s) of treatment sites during a treatment session.

12. A system for hair treatment comprising:
a laser module configured for generating a non-ablative laser beam;
a handpiece connected to the laser module configured to receive the non-ablative laser beam, and deliver a fractional pattern of the non-ablative laser beams to a treatment site on a person's scalp;
a controller adapted for controlling parameters of the non-ablative laser beam to apply the hair;
a non-transitory computer readable medium programmed with computer readable code that upon execution by the controller causes the controller to:
activate the laser module for a predetermined treatment time, at a predetermined pulse rate, and a predetermined pulse duration;
deliver the non-ablative laser to the treatment site on the person's scalp; and
deactivate the laser module.

13. The system of claim 12, wherein the laser module generates the non-ablative laser beam with a wavelength in the range of 1000-2000 nm.

14. The system of claim 13, wherein the laser module generates the non-ablative laser beams having at least one of the following wavelengths: 1940 nm, 1927 nm, 1565 nm, 1550 nm, 1540 nm, 1470 nm, 1450 nm, 1440 nm, 1410 nm, 1340 nm, 1320 nm, 1064 nm and 1060 nm.

15. The system of claim 12, further comprises a scanner configured define at least one of size and shape of a fractional pattern of non-ablative laser beams and delivers to the treatment site the fractional pattern of non-ablative laser beams comprising a plurality of spaced-apart micro-beams forming a certain shape that defines the treatment site.

16. The system of claim 15, wherein the plurality of spaced-apart micro-beams forms at least one of the following shapes: hexagon, doughnut, circle, square, horizontal rectangle, vertical rectangle, horizontal line, and vertical line.

17. The system of claim 16, wherein the plurality of spaced-apart micro-beams forms a circular shape having a diameter of up to 18 mm.

18. The system of claim 15, wherein the handpiece delivers the fractional pattern of non-ablative laser is at least one of: a density a range of 50 to 500 micro-beams per cm²; or an energy between 10-35 mJ per each micro-beam.

19. The system of claim 12, further comprises one more of tips configured to be mounted at a distal end of the handpiece, and the tips being configured to do at least one of the following:
define the shape of the treatment site;
form a spacer between a laser output of the handpiece and the treatment site;
move grown hair aside for treatment; or
conform to topography of the treatment site.

20. The system of claim 12, further comprises,
one more of cooling devices configured to maintain an optimal temperature of the handpiece or/and a tip mounted on a distal end of the handpiece;
temperature sensors;
the non-transitory computer readable medium programmed with computer readable code that upon execution by the controller causes the controller to:
automatically activate the one or more cooling devices if at least one of the following occurs:
the energy level is above a predetermined energy;
the density level is above a predetermined density; and
a temperature of the handpiece, a tip and/or the treatment site is above a predetermined temperature, based on the temperature sensors.
